SEQUENCE LISTING (2 sequences)

SEQ ID NO: 1 - protein, 1241 aa, Homo sapiens
MALGTTLRASLLLLGLLTEGLAQLAIPASVPRGFWALPENLTVVEGASVELRCGVSTPGSAVQWAKDGLLLGPDPRIPGFPRYRLEGDPARGEFHLHIEACDLSDDAEYECQVGRSEMGPELVSPRVILSILVPPKLLLLTPEAGTMVTWVAGQEYVVNCVSGDAKPAPDITILLSGQTISDISANVNEGSQQKLFTVEATARVTPRSSDNRQLLVCEASSPALEAPIKASFTVNVLFPPGPPVIEWPGLDEGHVRAGQSLELPCVARGGNPLATLQWLKNGQPVSTAWGTEHTQAVARSVLVMTVRPEDHGAQLSCEAHNSVSAGTQEHGITLQVTFPPSAIIILGSASQTENKNVTLSCVSKSSRPRVLLRWWLGWRQLLPMEETVMDGLHGGHISMSNLTFLARREDNGLTLTCEAFSEAFTKETFKKSLILNVKYPAQKLWIEGPPEGQKLRAGTRVRLVCLAIGGNPEPSLMWYKDSRTVTESRLPQESRRVHLGSVEKSGSTFSRELVLVTGPSDNQAKFTCKAGQLSASTQLAVQFPPTNVTILANASALRPGDALNLTCVSVSSNPPVNLSWDKEGERLEGVAAPPRRAPFKGSAAARSVLLQVSSRDHGQRVTCRAHSAELRETVSSFYRLNVLYRPEFLGEQVLVVTAVEQGEALLPVSVSANPAPEAFNWTFRGYRLSPAGGPRHRILSSGALHLWNVTRADDGLYQLHCQNSEGTAEARLRLDVHYAPTIRALQDPTEVNVGGSVDIVCTVDANPILPGMFNWERLGEDEEDQSLDDMEKISRGPTGRLRIHHAKLAQAGAYQCIVDNGVAPPARRLLRLVVRFAPQVEHPTPLTKVAAAGDSTSSATLHCRARGVPNIVFTWTKNGVPLDLQDPRYTEHTYHQGGVHSSLLTIANVSAAQDYALFTCTATNALGSDQTNIQLVSISRPDPPSGLKVVSLTPHSVGLEWKPGFDGGLPQRFCIRYEALGTPGFHYVDVVPPQATTFTLTGLQPSTRYRVWLLASNALGDSGLADKGTQLPITTPGLHQPSGEPEDQLPTEPPSGPSGLPLLPVLFALGGLLLLSNASCVGGVLWQRRLRRLAEGISEKTEAGSEEDRVRNEYEESQWTGERDTQSSTVSTTEAEPYYRSLRDFSPQLPPTQEEVSYSRGFTGEDEDMAFPGHLYDEVERTYPPSGAWGPLYDEVQMGPWDLHWPEDTYQDPRGIYDQVAGDLDTLEPDSLPFELRGHLV

SEQ ID NO: 2 - protein, 31 aa, Artificial Sequence
  Synthetic fusion protein
PEDQLPTEPPSGISEKTEAGSEEDRVRNEYE

(19) United States
(12) Patent Application Publication
(10) Pub. No.: US 2013/0101578 A1
(43) Pub. Date: Apr. 25, 2013

(54) BIOMARKER FOR RENAL FUNCTION IN PATIENTS WITH TYPE 2 DIABETES

(71) Applicants: Peng Keat Daniel Ng, Singapore (SG); Bee Choo Tai, Singapore (SG); Kee Seng Chia, Singapore (SG); Harry Holthofer, Helsinki (FI)

(72) Inventors: Peng Keat Daniel Ng, Singapore (SG); Bee Choo Tai, Singapore (SG); Kee Seng Chia, Singapore (SG); Harry Holthofer, Helsinki (FI)

(73) Assignee: NATIONAL UNIVERSITY OF SINGAPORE, Singapore (SG)

(21) Appl. No.: 13/640,991

(22) PCT Filed: Apr. 12, 2011
(86) PCT No.: PCT/SG11/00143

§ 371 Date: Oct. 12, 2012

Related U.S. Application Data
(60) Provisional application No. 61/322,949, filed on Apr. 12, 2010.

(51) Int. Cl. G01N 33/68; H01J 49/00; G01N 30/00

(52) U.S. Cl. CPC G01N 33/6893; G01N 30/00; H01J 49/004
USPC 424/130.1; 436/501; 435/7.92; 506/9; 435/287.2; 73/61.52; 250/282

(57) ABSTRACT

The invention provides kits and methods for the diagnosis, prognosis, and treatment of reduced kidney function in patients, such as diabetic patients. The methods include a step of detecting one or more nephrin degradation products in a sample from the subject, such as a urine sample.

### RELATED APPLICATION

This application claims the benefit of U.S. Provisional Application No. 61/322,949, filed on Apr. 12, 2010. The entire teachings of the above application are incorporated by reference.

### BACKGROUND OF THE INVENTION

The renal glomerulus plays a critical role in the modulation of albuminuria and renal function. In addition to its role in congenital nephrotic syndrome of the Finnish type, the importance of the transmembrane protein nephrin (NPHS1) in modulating renal phenotypes is also evidenced in diabetes. In models of experimental diabetes, dysregulation of nephrin gene expression has been associated with albuminuriaThe dominant and traditional view of the natural history of diabetic nephropathy is that albuminuria precedes a decline in renal function but this has been subject to recent debate (Retnakaran et al. 2006). It has been variously reported that a decline in renal function (expressed as the glomerular filtration rate, GFR) may be evident even in the absence of albuminuria (MacIsaac et al. 2004). The involvement of nephrin in controlling renal function is unclear, however, with the literature largely emphasizing the role of this protein in altered renal parameters reflected as albuminuria.

In view of the importance of kidney function in maintaining homeostasis and the prevalence of disorders associated with an increased risk of reduced renal function, such as type 2 diabetes, a need exists for methods for the diagnosis, prognosis, and treatment of reduced kidney function, as well as kits for performing such methods.

### SUMMARY OF THE INVENTION

The invention provides, inter alia, diagnostic and prognostic methods for detecting reduced renal function in a subject, as well as methods of treating a subject with reduced renal function and kits for performing any of these methods. The invention is based, at least in part, on the significant discovery that fragments of the nephrin protein are detectable in the urine of patients with type 2 diabetes are associated with reduced renal function and that the presence of one or more nephrin protein fragments are indicative of reduced renal function.

Accordingly, in one aspect, the invention provides methods of detecting reduced renal function in a mammalian subject by detecting one or more nephrin degradation products in a urine sample from the subject, where detection of one or more nephrin degradation products in the urine sample indicates reduced renal function in the subject. In particular embodiments, the subject is a human. In some embodiments, the subject is normoalbuminuric. In particular embodiments, the subject has or is at increased risk for developing type 2 diabetes (T2D). In more particular embodiments, the subject has been diagnosed with T2D for less than 1 year. In certain embodiments, the subject exhibits a decline in eGFR of at least 1.0 ml/min/1.73 m2.

In certain embodiments, the one or more nephrin degradation products has an apparent molecular weight of about 25 kDa, 50 kDa, 60 kDa, or 75 kDa and in more particular embodiments, an apparent molecular weight of about 25 kDa. The one or more nephrin degradation products can be detected by a variety of means including ELISA, Western Blotting, HPLC, SPR, SAT, aptamers, pepide sequencing, or MS/MS. In certain embodiments, the nephrin degradation products are detected using an antibody and in more particular embodiments, the antibody is a monoclonal antibody. In particular embodiments, the nephrin degradation products are detected in, a cell free fraction of a urine sample.

In another aspect, the invention provides methods of treating reduced kidney function. For example, the treatment methods of the invention can include the steps of any of the diagnostic methods provided by the invention with the further step(s) of administering a suitable prophylaxis for reduced renal function to the subject.

In yet another aspect, the invention provides kits for detecting reduced renal function in a mammal. The kits include can include any or all of the components for performing any of the methods provided by the invention. In some embodiments, the kits include an antibody that binds one or more nephrin degradation products. In more particular embodiments, the kit also includes instructions for use. In some embodiments, the kit includes one or more nephrin degradation products suitable for use as a positive control. In certain embodiments, the antibody in the kit is associated with an immunochromatographic device, such as a dip test.

The invention advantageously enables the skilled artisan to quickly and easily identify subjects with reduced renal function, including situations where the subject exhibits normal kidney function according to existing assays, such as albumin levels in the urine.

### DETAILED DESCRIPTION OF THE INVENTION

The invention provides kits and methods for the diagnosis, prognosis, and treatment of reduced kidney function in patients, such as diabetic patients. The methods include a step of detecting one or more nephrin degradation products in a sample from the subject, such as a urine sample. A description of example embodiments of the invention follows.

### Nephrin Degradation Products

Nephrin, the protein product of NPHS1, is a member of the immunoglobulin family of cell adhesion molecules that functions in the glomerular filtration barrier in the kidney. The gene is primarily expressed in renal tissues, and the protein is a type-1 transmembrane protein found at the slit diaphragm of glomerular podocytes. Nephrin genes have been identified in a variety of organisms, some of which are summarized in Table A, below.

TABLE A
Nephrin genes

| Organism | GeneID |
|---|---|
| Bos taurus | 519706 |
| Canis lupus familiaris | 484571 |
| Danio rerio | 692352 |
| Homo sapiens | 4868 |
| Mus musculus | 54631 |
| Oryctolagus cuniculus | 100354996 |
| Pan troglodytes | 468841 |
| Rattus norvegicus | 64563 |
| Xenopus laevis | 734213 |

The gene identifiers in Table A may be used to retrieve, inter alia, publicly-available annotated mRNA or protein sequences from sources such as the NCBI website, which may be found at the following uniform resource locator (URL): http://www.ncbi.nlm.nih.gov. The information associated with these identifiers, including reference sequences and their associated annotations, are incorporated by reference. For example, the gene identifiers can be used to retrieve human, mouse, and rat nephrin protein reference sequences and associated annotations (such as structural domains), such as NP_{—}004637 (SEQ ID NO: 1), NP_{—}062332.2, and NP_{—}062332.2, respectively. See also, HomoloGene: 20974, which provides multiple sequence alignment information for nephrin protein sequences from a variety of organisms. These sequence comparisons can be used to identify regions that are conserved between organisms and would therefore be expected to be important to the structure and/or function of nephrin proteins. Additional useful tools for converting IDs or obtaining additional information on a gene are known in the art and include, for example, DAVID, Clone/GeneID converter, and SNAD. See Huang et al., Nature Protoc. 4(1):44-57 (2009), Huang et al., Nucleic Acids Res. 37(1)1-13 (2009), Alibes et al., BMC Bioinformatics 8:9 (2007), Sidorov et al., BMC Bioinformatics 10:251 (2009).

“Nephrin degradation product(s)” are fragments of nephrin protein that correspond to fragments detectable in the urine of human subjects with reduced kidney function where the nephrin degradation products in human exhibit apparent molecular weights of 25, 50, 60 or 75 kDa on an SDS-PAGE Western Blot under reducing conditions. “Corresponds to” means an analogous sequence from another organism as determined by suitable means known in the art, such as sequence alignments. For example, a corresponding nephrin degradation product from a non-human animal may exhibit apparent molecular weights that are identical, highly similar (<=10% deviation-higher or lower—e.g., 9, 8, 7, 6, 5, 4, 3, 2, 1%, or less), similar (10-20% deviation), or dissimilar (>20% deviation, e.g., 20, 25, 30, 35, 40, 50, 60, 70, 80, 90, 100%, or more) to human. In certain embodiments, a nephrin degradation product includes a sequence at least 60, 65, 70, 75, 80, 85, 90, 95, 96, 97, 98, 99%, or more identical to a fragment of at least 5, 10, 12, 13, 14, 15, 16, 17, 18, 19, 20, 50, 100, 200, 400, 600, 800, or 1000 contiguous amino acids of SEQ ID NO: 1, the reference human nephrin protein sequence with NCBI accession number NP_{—}004637, which is incorporated by reference in its entirety. For example, in particular embodiments, a nephrin degradation product may include an amino acid sequence that is at least 60, 65, 70, 75, 80, 85, 90, 95, 96, 97, 98, 99% identical to the sequence of human nephrin degradation products that exhibit apparent molecular weights of 25, 50, 60, or 75 kDa on an SDS-PAGE Western Blot under reducing conditions. These sequences may be determined using routine methods in the art, such as, for example, peptide sequencing of fractionated samples, e.g., following Western Blotting or two or higher dimensional protein electrophoresis.

Programs for sequence alignments and comparisons include FASTA (Lipman and Pearson, Science, 227: 1435-41 (1985) and Lipman and Pearson, PNAS, 85: 2444-48), BLAST (McGinnis & Madden, Nucleic Acids Res., 32:W20-W25 (2004) (current BLAST reference, describing, inter alia, MegaBlast); Zhang et al., J. Comput. Biol., 7(1-2):203-14 (2000) (describing the “greedy algorithm” implemented in MegaBlast); Altschul et al., J. Mol. Biol., 215:403-410 (1990) (original BLAST publication)), Needleman-Wunsch (Needleman and Wunsch, J. Molec. Bio., 48 (3): 443-53 (1970)), Sellers (Sellers, Bull. Math. Biol., 46:501-14 (1984), and Smith-Waterman (Smith and Waterman, J. Molec. Bio., 147: 195-197 (1981)), and other algorithms (including those described in Gerhard et al., Genome Res., 14(10b):2121-27 (2004)), which are incorporated by reference. In particular embodiments, sequences are compared by BLAST using default parameters for protein queries.

### Subjects

Subjects to be diagnosed, prognosed, or treated by the methods and/or kits provided by the invention can be any mammal, such as a primate, a rodent, a canine, a feline, a porcine, an ovine, a bovine, or a leporine. In still more particular embodiments, the subject is a primate, e.g., a human.

The subject may be male or female and at any stage of development, e.g., a fetus, neonate, infant, child, adolescent, adult, or geriatric. In particular embodiments, the subject is a child, adolescent, adult, or geriatric. In still more particular embodiments, the subject is an adult or geriatric.

Subjects for the methods provided by the invention can be defined by a variety of clinical parameters. In particular embodiments, the subject may be at increased risk for or have diabetes, e.g., type 1 (T1D) or type 2 (T2D) diabetes and in more particular embodiments, the subject has or is at increased risk for developing T2D, for example, a subject may be a borderline T2D subject, e.g., impaired glucose tolerance or impaired fasting glycemia. A subject that has T2D, impaired fasting glucose, impaired glucose tolerance, can be defined according to the criteria in Table B:

TABLE B
where 2 hour glucose is 2 hours after ingestion of 75 g oral glucose.

| Condition | Test | Classifier |
|---|---|---|
| Diabetes | Fasting plasma | ≧7.0 mmol/l (126 mg/dl) |
| | glucose | or |
| | or | ≧11.1 mmol/l (200 mg/dl) |
| | 2-h plasma | or |
| | glucose | ≧11.1 mmol/l (200 mg/dl) |
| | or | or |
| | Causal plasma | ≧6.5% |
| | glucose | |
| | or | |
| | HbA1c | |
| Impaired glucose | 2-h plasma | ≧7.8 and <11.1 mmol/l |
| tolerance | glucose | (140 mg/dl and 200 mg/dl) |
| Impaired fasting | Fasting plasma | 6.1 to 6.9 mmol/l |
| glucose | glucose | (110 mg/dl to 125 mg/dl) (WHO |
| | | guideline) |
| | | or |
| | | 5.6 to 6.9 mmol/l |
| | | (100 mg/dl to 125 mg/dl) |
| | | (ADA guideline) |

Diabetic subjects may have had diabetes (T1D or T2D) for any period of time, such as at least 1, 6, or 12 months, or 1, 2, 3, 4, 5, 6, 7, 8, 9, 10, 15, 20, 25 years, or more. In particular embodiments, the subject has been diagnosed as diabetic for less than 1 year, e.g., less than 12, 11, 10, 9, 8, 7, 6, 5, 4, 3, 2, or 1 months. The subject may be normoalbuminuric or have varying levels of albuminuria. A “normoalbuminuric” subject, or subject with “normoalbuminuria” and the like, exhibits clinically normal levels of albumin protein in the urine, consistent with normal kidney function. Albuminuria status can be determined by any means known in the art and, in some embodiments, can be characterized by albumin creatine ratio (ACR). For example, in certain embodiments, a normoalbuminuric subject has an ACR of less than about 30 mg/g, e.g., less than 32, 30, 28, 26, 24, 22, 20, 10, 15, 10, 5 mg/g, or less. Subjects may have any level of kidney function, which may be measured by any means, such as blood urea nitrogen, glomular filtration rate, inulin assay, or estimated glomular filtration rate (eGFR). Kidney function may be at 100, 95, 90, 85, 80, 70, 60, 50, 40, 30, 20, 10%, or less, of normal levels as measured by any of these assays. A normal range for eGFR is 60 to 90 ml/min/1.73 m², although it may exceed 90 ml/min/1.73 m² in some individuals. Accordingly, in some embodiments, a subject may exhibit reduced eGFR of at least 1, 2, 4, 6, 8, 10, 12, 14, 16, 18, 20, 22 ml/min/1.73 m², or more. For example, in certain particular embodiments, a subject may exhibit an eGFR of less than 65, 64, 63, 62, 61, 60, 59, 58, 57, 56, 55, 54, 53, 52, 51, 50, 45, 40, 35, 30 ml/min/1.73 m², or less. T2D subjects treated by the method provided by the invention may be concurrently, previously, or subsequently treated by methods for regulating T2D, such as, modified diet, or administration of insulin, metformin, sulfonylureas, nonsulfonylurea secretagogues, alpha glucosidase inhibitors, thiazolidinediones, or combinations thereof.

Suitable subjects can be at any body mass index (kg/m²), e.g., <16 (severely underweight), 16-18.5 (underweight), 18.5-25 (normal), 25-30 (overweight), 30-35 (obese I), 35-40 (obese II) or >40 (obese III). In particular embodiments, the subject may have a BMI of >25. In certain embodiments, a subject may exhibit a waist to hip ratio (WHR) of about 0.30, 0.35, 0.40, 0.45, 0.50, 0.55, 0.60, 0.65, 0.70, 0.75, 0.80, 0.85, 0.90, 0.95, or 1.00. In some embodiments, a subject may exhibit triglyceride levels of about 0.9, 1.0, 1.1, 1.2, 1.3, 1.4, 1.5, 1.6, 1.7, 1.8, 1.9, 2.0, or 2.1 (mmol/L) or In triglyceride levels of about −2, −3, −4, −5, −6, −7, −8, −9, −10, −11, −12, −13, or −14.

Subjects may have any combination of the above parameters. A subject tested by the methods provided by the invention may exhibit 0, 1, 2, 3 or 4 nephrin degradation products. In particular embodiments, a subject determined to have reduced kidney function by the methods provided by the invention exhibits at least 1, 2, 3 or 4 nephrin degradation products in a urine sample. In more particular embodiments, a human subject determined to have reduced kidney function by the methods of the invention exhibits, in a urine sample, at least one of the nephrin degradation products with apparent molecular weights of 25 kDa, 50 kDa, 60 kDa or 75, kDa on an SDS-PAGE Western Blot under reducing conditions.

### Kits and Methods

The methods provided by the invention include the step of detecting nephrin degradation products—determining the presence of nephrin degradation products indicates reduced renal function in a subject. Nephrin degradation products may be detected by any means of protein detection known in the art, including, for example, ELISA, Western Blotting, RIA (radioimmunoassay), nucleic acid-based or protein-based aptamer techniques, HPLC (high precision liquid chromatography), SPR (surface plasmon resonance), SAT (suspension array technology—including both immune-based, aptamer-based, or combination methods), direct peptide sequencing (such as Edman degradation sequencing), or mass spectrometry (such as MS/MS). In particular embodiments, nephrin degradation products are detected using an antibody that binds one or more nephrin degradation products. In still more particular embodiments, the antibody binds the sequence pedqlptepp sgisekteag seedrvrney e (SEQ ID NO: 2), which corresponds to a fusion of amino acids 1045-1056 and 1097-1115 of SEQ ID NO: 1. In other embodiments, the antibody binds to a sequence comprising an amino acid sequence at least 60, 65, 70, 75, 80, 85, 90, 95, 96, 97, 98, 99, or 100% identical to SEQ ID NO: 2 or a fragment thereof that is at least 10, 12, 13, 15, 18, 19, 20, 25, or 30 amino acids in length. For example, in more particular embodiments, the antibody binds to a sequence that is at least 60, 65, 70, 75, 80, 85, 90, 95, 96, 97, 98, 99, or 100% identical to amino acids 1 to 12 of SEQ ID NO: 2, while in other particular embodiments, the antibody binds to a sequence that is at least 60, 65, 70, 75, 80, 85, 90, 95, 96, 97, 98, 99, or 100% identical to amino acids 13-31 of SEQ ID NO: 2. In certain embodiments, antibodies for use in the methods and kits provided by the invention bind any of the foregoing antigens with a Ka of greater than about 1.0×10⁶, 5.0×10⁶, 1.0×10⁷, 5.0×10⁷, 1.0×10⁸, 5.0×10⁸, 1.0×10⁹ M⁻¹, or more.

The term “antibody,” as used herein, refers to any polypeptide comprising an antigen-binding site regardless of the source, species of origin, method of production, and characteristics. In particular embodiments, an antibody is an immunoglobulin or an antigen-binding fragment thereof. As a non-limiting example, the term “antibody” includes human, orangutan, macaque, camel, mouse, rat, rabbit, goat, sheep, and chicken antibodies. The term includes but is not limited to polyclonal, monoclonal, monospecific, polyspecific, non-specific, humanized, camelized, single-chain, chimeric, synthetic, recombinant, hybrid, mutated, and CDR-grafted antibodies. For the purposes of the present invention, it also includes, unless otherwise stated, antibody fragments such as Fab, F(ab′)2, Fv, scFv, Fd, dAb, VHH (also referred to as nanobodies), and other antibody fragments that retain the antigen-binding function. Antibodies also include antigen-binding molecules that are not based on immunoglobulins, as further described below.

Antibodies to nephrin can be made, for example, via traditional hybridoma techniques (Kohler and Milstein, Nature 256: 495-499 (1975)), recombinant DNA methods (U.S. Pat. No. 4,816,567), or phage display techniques using antibody libraries (Clackson et al., Nature 352: 624-628 (1991); Marks et al., J. Mol. Biol. 222: 581-597 (1991)). For various other antibody production techniques, see Antibodies: A Laboratory Manual, eds. Harlow et al., Cold Spring Harbor Laboratory, 1988.

In some embodiments, the term “antibody” includes an antigen-binding molecule based on a scaffold other than an immunoglobulin. For example, non-immunoglobulin scaffolds known in the art include small modular immunopharmaceuticals (see, e.g., U.S. Patent Application Publication Nos. 2008/0181892 and 2008/0227958 published Jul. 31, 2008 and Sep. 18, 2008, respectively), tetranectins, fibronectin domains (e.g., AdNectins, see U.S. Patent Application Publication No. 2007/0082365, published Apr. 12, 2007), protein A, lipocalins (see, e.g., U.S. Pat. No. 7,118,915), ankyrin repeats, and thioredoxin. Molecules based on non-immunoglobulin scaffolds are generally produced by in vitro selection of libraries by phage display (see, e.g., Hoogenboom, Method Mol. Biol. 178:1-37 (2002)), ribosome display (see, e.g., Hanes et al., FEBS Lett. 450:105-110 (1999) and He and Taussig, J. Immunol. Methods 297:73-82 (2005)), or other techniques known in the art (see also Binz et al., Nat. Biotech. 23:1257-68 (2005); Rothe et al., FASEB J. 20:1599-1610 (2006); and U.S. Pat. Nos. 7,270,950; 6,518,018; and 6,281,344) to identify high-affinity binding sequences.

In particular embodiments, the methods of the invention include the step of detecting nephrin degradation products in a sample from a subject by Western Blot using a nephrin antibody that recognizes nephrin degradation products. The sample from the subject may be, for example, a blood or urine sample, and in particular embodiments is a urine sample. The sample may be prepared by any means suitable for the particular detection method employed. For example, in particular embodiments, a urine sample may be precipitated, for example, with trichloroacetic acid, centrifuged, optionally washed, and then resuspended in a suitable volume of buffer, such as Laemmli, before analysis. Pre-analysis may also include heating, for example, at about 60, 70, 80, 90, or 95° C. for a sufficient period of time, e.g., about 1, 2, 3, 4, 5, 10, 15, or more minutes. In particular embodiments, a urine sample to be analyzed by the methods provided by the invention is provided in a volume sufficient to contain at least 10, 15, 20, 25, 30, 35 μg, or more, of protein. In particular embodiments, the urine sample provides approximately 30 μg of protein. In certain particular embodiment, a urine sample may be processed before precipitation, e.g., the sample is briefly centrifuged to produce a substantially cell-free sample for analysis. The sample may be frozen for later analysis or processed immediately.

Treatment methods provided by the invention may include the steps of any of the diagnostic methods described in the application and further include the step of providing a suitable prophylaxis to a subject found to have reduced kidney function. Suitable prophylaxis for reduced kidney function are known in the art and include, modified diet, modified activity schedule, modified fluid intake, modified osmolyte (e.g., salt) intake (e.g., a reduction of 5, 10, 15, 20, 30, 40, 50, 60, 70, 80, 90, 95% or more of the USRDA of salt intake), administering antagonists of the renin-angiotensin system (RAS)/renin-angiotensin-aldosterone system (RAAS) pathway, dialysis, kidney transplant, or any combination of the foregoing.

Examples of RAS pathway antagonists are known in the art and include inhibitors of various components of the RAS pathway, including e.g., antibodies or vaccines, dominant negative proteins, aptamers, and small molecule antagonists of RAS pathway components, including combinations thereof. Components of the RAS pathway and specific inhibitors are known and include renin inhibitors (e.g., aliskiren, remikiren, and enalkiren), ACE (angiotensin-converting enzyme) inhibitors (e.g., benazepril, captopril, enalapril, fosinopril, imidapril, lisinopril, perindopril, quinapril and ramipril, zofenopril, as well as casokinins, lactokinins and lactopeptides, such as Val-Pro-Pro or Ile-Pro-Pro), inhibitors of angiotensin II signaling (e.g., antagonists of its receptors; particular AngII receptor antagonists include azilsartan, candesartan, eprosartan, exp 3174, irbesartan, losartan, olmesartan, telmisartan, and valsartan). In more particular embodiments RAS-pathway inhibitors can prevent formation of renal fibrosis. In some embodiments, renal fibrosis can be prevented or ameliorated by additional method known in the art, such as antagonism of TGF-β activity, for example, by small molecules, antibodies, or receptor decoys, such as receptor-Fc fusions.

The kits provided by the invention may include some or all of the components needed to perform any of the methods of the invention. For example, in certain embodiments, a kit includes a nephrin antibody that recognizes nephrin degradation products. The kit may include further elements, such as instructions for use, and/or suitable controls, such as one or more nephrin degradation products. In certain embodiments, the antibody in the kit is associated with a specialized device, such as a dip test lateral flow device (e.g., an immunochromatographic device), as well as immunomagnetic devices and non-immune-based devices, such as aptamer or small-molecule based devices. In particular embodiments, the devices provided in the kits may be one-time use devices.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers, including, for example, genomic loci, genomic sequences, functional annotations, allelic variants, and reference mRNA (including, e.g., exon boundaries or response elements) and protein sequences (such as conserved domain structures and/or mature regions of a preprotein) are hereby incorporated by reference in their entirety.

### EXEMPLIFICATION

### Materials and Methods

### Patients and Urine Samples

This study was conducted on 381 Chinese patients with type 2 diabetes who were recruited as part of the Singapore Diabetes Cohort Study (SDCS) (Unoki et al 2008, Ng et al 2008). Mid-stream spot urine samples were collected and transported chilled to the laboratory, centrifuged and stored at −80° C. prior to analysis. Albuminuria was determined on the basis of ACRs (mg/g) using commercially available kits for albumin and creatinine measurements (Exocell, Philadelphia, Pa.). Renal function (expressed as GFR) was estimated using the simplified Modification of Diet in Renal Disease (MDRD) equation where the estimated GFR (eGFR, ml/min/1.73 m²)=186.3×(plasma creatinine in mg/dL)^{-1.154}×(age in years)^{-0.203}×(0.742 for women)×(1.21 if subject is black) [NKF 2002].

### Nephrin Analysis and Western Blotting

For nephrin analysis, urine sample volumes corresponding to a total protein quantity of 30 ug were precipitated with 10% (wt/vol) trichloroacetic acid in PBS on ice for 30 min. Thereafter the samples were centrifuged for 10 min at 13000 g at +4° C. and the precipitate washed in ice-cold acetone. The samples were then air-dried and dissolved in Laemmli buffer (62.5 mmol/l Tris-HCl, 10% glycerol, 2% SDS, 5% 2-mercaptoethanol and 0.05% bromophenol blue) and heated at 95° C. for 5 min. The samples were analyzed in 10% polyacrylamide gels with the Protean Mini-gel electrophoresis system using ready gels (Biorad Laboratories, Hercules, Calif.). A nephrinuric urine sample from a patient with type I diabetes was used as positive control in every gel. After electrophoresis, the proteins were transferred onto nitrocellulose filters (Amersham Biosciences, Buckinhamshire, UK) and blocked for 2 hours at room temperature with 3% non-fat dried milk (Valio, Helsinki, Finland) in PBS. Thereafter the filters were soaked in primary anti-nephrin antibody in PBS containing 1% milk as above and 0.02% sodium azide at room temperature, followed by thorough washes in PBS containing 0.2% Tween 20. The filters were then incubated with horseradish peroxidase-labeled second antibodies for one hour at room temperature and washed as above. The bound antibody was detected with Super Signal ECL substrate (Pierce, Rockford, Ill.). The presence of protein bands with the antibody staining was regarded as positive for nephrinuria. The typical prominent bands were detected at 25, 50, 60 and 75 kDa areas with variable intensities and individual expression patterns as compared with the standards ladder.

### Statistical Analysis

The Student's t-test was used to compare differences in means between two groups of interest. For skewed data, the non-parametric equivalent based on the Wilcoxon rank sum test was used instead. The associations between the presence of immuno-reactive nephrin fragments in urine samples were evaluated using the Pearson's χ² test. To investigate whether there is any association between nephrinuria and renal function, while taking albuminuric status into account, multiple linear regression analysis was implemented. Log transformation was carried out on skewed data such ACR, triglycerides and diabetes duration prior to the multiple linear regression analysis. The regression analyses were adjusted for potential confounders such as log triglycerides, waist hip ratio and age, where appropriate. All statistical evaluations were assessed based on a two-sided test at the conventional 5% level using STATA (version 10).

### Results

The main clinical characteristics of the 381 patients are presented as stratified by gender (Table 1). Males have larger WHR (P<0.001), lower levels of both LDL (P=0.002) and HDL cholesterol (P<0.001) compared to females. Western blot analysis of the urine samples using a monoclonal antibody against human nephrin revealed the presence of up to four distinct protein bands with approximate molecular weights of 25, 50, 60, and 75 kDa. The most prevalent fragment was that with a molecular weight of 60 kDa which, occurred in 155 of 381 samples (40.7%). This was followed by the fragments at 50 kDa (130/381, 34.1%), 25 kDa (88/381, 23.1%), and lastly, 75 kDa (59/381, 15.4%). The presence of these fragments was highly correlated with each other as seen in cross-tabulations (P<0.001 for all comparisons) (Supplementary Table 1).

In multivariate analyses, the presence of each of the four fragments was associated with a decline in eGFR (largest P-value=0.003) (Table 2). Logarithmic form of ACR (ln ACR) did not emerge as a significant independent predictor of eGFR (Model 1 in Tables 2). Even with the deliberate retention of ln ACR (Model 2 in Table 2), the presence of either the 25, 50 and 60 kDa fragments remained significantly associated with a loss of eGFR (all P values <0.05). The exception was the 75 kDa fragment which while showing a trend towards a loss of eGFR, did not reach statistical significance (Table 2). Inclusion of ln ACR did not improve the overall fit of the regression model in predicting eGFR based on R² values (Table 2). The association of a lower eGFR with nephrinuria therefore was not confounded by the albuminuric status. WHR and ln triglyeride levels emerged as significant covariates. Age was expectedly associated with eGFR since it was used in the MDRD equation for the computing this variable (Table 2).

Among the four, the 25 kDa fragment was the strongest predictor of eGFR especially after adjustment for ln ACR (Tables 2). The presence of this fragment was associated with a loss in eGFR of 6.55 ml/min/1.73 m²(95% confidence interval: 0.07 to 13.03). Stratifying the patient samples according to the presence of the 25 kDa fragment did not reveal any significant differences in clinical characteristics except for eGFR which was consistent with the multivariate analysis. Diabetes duration and WHR were greater in the presence of the 25 kDa fragment but these were only of borderline significance (Supplementary Table 2).

We next sought to characterize the potential association of nephrinuria with eGFR among 228 patients with normoalbuminuria as defined as having an ACR<=30 mg/g. In multivariate analyses, nephrinuria with the exception of the 75 kDa fragment was significantly associated with a loss of eGFR (Table 3). Particularly, the presence of the 25 kDa fragment was associated with a loss in eGFR of 17.29 (95% CI: 6.56 to 28.01) ml/min/1.73 m² after adjustment for ln ACR (P=0.002) (Table 3). In triglycerides and WHR emerged as significant independent predictors of eGFR (Tables 3). Stratifying the patient samples according to the presence of the 25 kDa fragment among the normoalbuminuric patients did not reveal any significant differences in clinical characteristics except for eGFR. ACR was slightly higher in the presence of the 25 kDa fragment but this did not reach formal statistical significance (P=0.077) (Supplementary Table 3).

### Discussion

In our current study, 5.3% of normoalbuminuric patients with type 2 diabetes showed evidence of nephrinuria as based on the presence of the 25 kDa fragment. Our study has also provided clinical information that supports this novel role of nephrin in modulating renal function. This positive finding was not only independent of albuminuria, but notably, it gained further statistical strength when the study was confined to normoalbuminuric diabetic patients.

Our finding has raised an intriguing biological question as to whether there is already early damage to the SD even in these normoalbuminuric patients. Our results clearly show that a subset of these patients present with both nephrin fragments and albumin early on during their disease course as measurable in the urine. For However, subsequent uptake of albumin at the proximal tubules by the specific endocytotic mechanism employing the megalin-cubulin protein complex could have delayed the clinical appearance of microalbuminuria. Consistent with this notion, a recent proteomic study reported that enhanced excretion of megalin and cubulin in the urine (possibly reflecting damage to the proximal tubular uptake mechanism) was associated with the appearance of microalbuminuria in type 1 diabetic patients (Thrailkill 2009). In contrast to albumin reabsorption, it is much less clear whether nephrin fragments that have been shed into the urinary space early on could be actively taken up at the proximal tubules.

It would seem plausible that early damage to the podocytes, as indicated by nephrinuria, could potentially impact glomerular filtration. In conclusion, nephrinuria may provide new clinical insights into renal biology in diabetes even in normoalbuminuric patients who have traditionally been perceived as having a low risk of chronic kidney disease.

### REFERENCES

1) Retnakaran et al., Diabetes 55, 1832-1839, 2006
2) MacIsaac et al., Diabetes Care 27, 195-200, 2004
3) National Kidney Foundation (NKF 2002) K/DOQI clinical practice guidelines for chronic kidney disease: evaluation, classification, and stratification. Am J Kidney Dis, 39, S1-266, 2002
4) Ng et al., Diabetologia, 51, 2318-2324, 2008
5) Unoki et al., Nature Genetics, 40, 1098-1102, 2008
6) Thrailkill et al., Diabetes Care, 32, 1266-1268, 2009

TABLE 1
Characteristics of Chinese Type 2 diabetic patients according to gender

| | Total | | Male | | Female | | |
|---|---|---|---|---|---|---|---|
| | (n = 381) | | (n = 183) | | (n = 198) | | p-value |
| Age, year | 65.7 | (9.3) | 65.3 | (9.30) | 66.1 | (9.35) | 0.445 |
| Diabetes duration, yr | 6 | (2-15) | 6 | (2-15) | 5.5 | (3-15) | 0.947 |
| BMI, kg/m² | 25.64 | (4.36) | 25.70 | (4.40) | 25.59 | (4.34) | 0.801 |
| WHR | 0.91 | (0.07) | 0.95 | (0.06) | 0.88 | (0.05) | <0.001 |
| HbA1c, % | 7.19 | (0.99) | 7.28 | (0.99) | 7.11 | (1.00) | 0.107 |
| Systolic BP, mmHg | 132.6 | (12.15) | 132.0 | (12.58) | 133.2 | (11.67) | 0.334 |
| Diastolic BP, mmHg | 78.4 | (6.70) | 78.8 | (6.85) | 78.0 | (6.54) | 0.287 |
| Triglycerides, mmol/L | 1.3 | (1.0-1.9) | 1.3 | (0.9-1.8) | 1.4 | (1.0-2) | 0.272 |
| LDL cholesterol, mmol/L | 3.05 | (0.88) | 2.91 | (0.90) | 3.20 | (0.85) | 0.002 |
| HDL cholesterol, mmol/L | 1.23 | (0.35) | 1.10 | (0.31) | 1.36 | (0.35) | <0.001 |
| ACR, mg/g | 22 | (8.9-61.2) | 19.4 | (8.2-58.1) | 23.6 | (10.1-64.4) | 0.281 |
| eGFR, ml/min/1.73 m² | 81.9 | (25.2) | 79.7 | (23.88) | 84.3 | (26.4) | 0.083 |

Data are presented as mean (SD) or median (interquartile range).

TABLE 2
Association of eGFR with the presence of individual immuno-reactive nephrin
fragments in 381 Chinese diabetic patients

| | | | | Standardized | | |
|---|---|---|---|---|---|---|
| | | Estimate (95% CI) | | estimate | P-value | R² |
| | Model 1 | | | | | |
| | (without ln ACR) | | | | | |
| 25 kDa fragment | 25 kDa fragment | −8.84 | (−14.05 to −3.63) | −0.148 | 0.001 | 32.12 |
| | ln triglycerides | −8.33 | (−12.52 to −4.14) | −0.175 | <0.001 | |
| | WHR | −59.80 | (−93.33 to −26.27) | −0.158 | <0.001 | |
| | Age | −1.28 | (−1.53 to −1.04) | −0.458 | <0.001 | |
| | Model 2 | | | | | |
| | (including ln ACR) | | | | | |
| | 25 kDa fragment | −6.55 | (−13.03 to −0.07) | −0.110 | 0.047 | 31.42 |
| | ln ACR | −0.96 | (−2.60 to 0.67) | −0.065 | 0.245 | |
| | ln triglycerides | −8.06 | (−12.27 to −3.85) | −0.169 | <0.001 | |
| | WHR | −59.18 | (−92.71 to −25.66) | −0.156 | 0.001 | |
| | Age | −1.27 | (−1.52 to −1.03) | −0.455 | <0.001 | |
| | Model 1 | | | | | |
| | (without ln ACR) | | | | | |
| 50 kDa fragment | 50 kDa fragment | −7.63 | (−12.22 to −3.03) | −0.144 | 0.001 | 32.03 |
| | ln triglycerides | −8.26 | (−12.46 to −4.07) | −0.173 | <0.001 | |
| | WHR | −63.37 | (−96.79 to −29.96) | −0.167 | <0.001 | |
| | Age | −1.31 | (−1.55 to −1.06) | −0.466 | <0.001 | |
| | Model 2 | | | | | |
| | (including ln ACR) | | | | | |
| | 50 kDa fragment | −5.67 | (−10.87 to −0.48) | −0.107 | 0.032 | 32.50 |
| | ln ACR | −1.19 | (−2.67 to 0.30) | −0.080 | 0.116 | |
| | ln triglycerides | −7.94 | (−12.15 to −3.74) | −0.167 | <0.001 | |
| | WHR | −61.36 | (−94.80 to −27.92) | −0.162 | <0.001 | |
| | Age | −1.29 | (−1.53 to −1.04) | −0.459 | <0.001 | |
| | Model 1 | | | | | |
| | (without ln ACR) | | | | | |
| 60 kDa fragment | 60 kDa fragment | −7.53 | (−12.03 to −3.03) | −0.148 | 0.001 | 32.06 |
| | ln triglycerides | −7.59 | (−11.81 to −3.37) | −0.159 | <0.001 | |
| | WHR | −63.58 | (−96.98 to −30.17) | −0.167 | <0.001 | |
| | Age | −1.27 | (−1.51 to −1.02) | −0.452 | <0.001 | |
| | Model 2 | | | | | |
| | (including ln ACR) | | | | | |
| | 60 kDa fragment | −5.54 | (−10.86 to −0.21) | −0.109 | 0.042 | 32.42 |
| | ln ACR | −1.08 | (−2.63 to 0.47) | −0.072 | 0.172 | |
| | ln triglycerides | −7.48 | (−11.69 to −3.26) | −0.157 | 0.001 | |
| | WHR | −61.75 | (−95.21 to −28.28) | −0.163 | <0.001 | |
| | Age | −1.26 | (−1.50 to −1.01) | −0.449 | <0.001 | |
| | Model 1 | | | | | |
| | (without ln ACR) | | | | | |
| 75 kDa fragment | 75 kDa fragment | −9.11 | (−15.18 to −3.04) | −0.132 | 0.003 | 31.66 |
| | ln triglycerides | −7.76 | (−11.99 to −3.54) | −0.163 | <0.001 | |
| | WHR | −63.21 | (−96.72 to −29.70) | −0.166 | <0.001 | |
| | Age | −1.29 | (−1.54 to −1.05) | −0.461 | <0.001 | |
| | Model 2 | | | | | |
| | (including ln ACR) | | | | | |
| | 75 kDa fragment | −5.74 | (−13.47 to 1.99) | −0.083 | 0.145 | 32.03 |
| | ln ACR | −1.18 | (−2.85 to 0.50) | −0.079 | 0.168 | |
| | ln triglycerides | −7.64 | (−11.87 to −3.42) | −0.160 | <0.001 | |
| | WHR | −61.49 | (−95.05 to −27.93) | −0.162 | <0.001 | |
| | Age | −1.28 | (−1.52 to −1.04) | −0.457 | <0.001 | |

TABLE 3
Association of eGFR with the presence of individual immuno-reactive nephrin
fragments among 228 patients with normoalbuminuria (ACR <= 30 mg/g)

| | | | | Standardized | | |
|---|---|---|---|---|---|---|
| | | Estimate (95% CI) | | estimate | P-value | R² |
| | Model 1 | | | | | |
| | (without ln ACR) | | | | | |
| 25 kDa fragment | 25 kDa fragment | −16.58 | (−27.31 to −5.86) | −0.164 | 0.003 | 41.65 |
| | ln triglycerides | −7.80 | (−12.63 to −2.98) | −0.172 | 0.002 | |
| | WHR | −75.78 | (−111.97 to −39.59) | −0.221 | <0.001 | |
| | Age | −1.32 | (−1.58 to −1.06) | −0.532 | <0.001 | |
| | Model 2 | | | | | |
| | (including ln ACR) | | | | | |
| | 25 kDa fragment | −17.29 | (−28.01 to −6.56) | −0.171 | 0.002 | 42.31 |
| | ln ACR | 2.62 | (−0.74 to 5.97) | 0.082 | 0.126 | |
| | ln triglycerides | −7.67 | (−12.49 to −2.86) | −0.169 | 0.002 | |
| | WHR | −75.59 | (−111.66 to −39.52) | −0.221 | <0.001 | |
| | Age | −1.34 | (−1.60 to −1.08) | −0.540 | <0.001 | |
| | Model 1 | | | | | |
| | (without ln ACR) | | | | | |
| 50 kDa fragment | 50 kDa fragment | −5.72 | (−11.75 to −0.32) | −0.100 | 0.064 | 40.04 |
| | ln triglycerides | −7.14 | (−12.00 to −2.28) | −0.157 | 0.004 | |
| | WHR | −79.58 | (−116.27 to −42.89) | −0.232 | <0.001 | |
| | Age | −1.36 | (−1.62 to −1.09) | −0.547 | <0.001 | |
| | Model 2 | | | | | |
| | (including ln ACR) | | | | | |
| | 50 kDa fragment | −6.22 | (−12.28 to −0.16) | −0.109 | 0.044 | 40.67 |
| | ln ACR | 2.55 | (−0.86 to 5.97) | 0.080 | 0.142 | |
| | ln triglycerides | −7.00 | (−11.85 to −2.14) | −0.154 | 0.005 | |
| | WHR | −79.63 | (−116.22 to −43.04) | −0.232 | <0.001 | |
| | Age | −1.38 | (−1.64 to −1.11) | −0.555 | <0.001 | |
| | Model 1 | | | | | |
| | (without ln ACR) | | | | | |
| 60 kDa fragment | 60 kDa fragment | −7.50 | (−13.30 to −1.69) | −0.137 | 0.012 | 40.88 |
| | ln triglycerides | −6.38 | (−11.22 to −1.54) | −0.141 | 0.010 | |
| | WHR | −79.13 | (−115.54 to −42.72) | −0.231 | <0.001 | |
| | Age | −1.31 | (−1.58 to −1.05) | −0.530 | <0.001 | |
| | Model 2 | | | | | |
| | (including ln ACR) | | | | | |
| | 60 kDa fragment | −8.49 | (−14.37 to −2.61) | −0.156 | 0.005 | 41.77 |
| | ln ACR | 3.09 | (−0.33 to 6.51) | 0.097 | 0.076 | |
| | ln triglycerides | −6.12 | (−10.94 to −1.29) | −0.135 | 0.013 | |
| | WHR | −79.17 | (−115.38 to −42.95) | −0.231 | <0.001 | |
| | Age | −1.33 | (−1.59 to −1.07) | −0.537 | <0.001 | |
| | Model 1 | | | | | |
| | (without ln ACR) | | | | | |
| 75 kDa fragment | 75 kDa fragment | 3.54 | (−14.28 to 21.37) | 0.021 | 0.696 | 39.08 |
| | ln triglycerides | −6.93 | (−11.82 to −2.03) | −0.153 | 0.006 | |
| | WHR | −78.71 | (−115.81 to −41.61) | −0.230 | <0.001 | |
| | Age | −1.36 | (−1.63 to −1.10) | −0.551 | <0.001 | |
| | Model 2 | | | | | |
| | (including ln ACR) | | | | | |
| | 75 kDa fragment | −2.40 | (−15.50 to 20.30) | −0.015 | 0.792 | 39.51 |
| | ln ACR | −2.12 | (−1.33 to 5.56) | −0.066 | 0.227 | |
| | ln triglycerides | −6.79 | (−11.68 to −1.89) | −0.149 | 0.007 | |
| | WHR | −78.42 | (−115.49 to −41.36) | −0.229 | <0.001 | |
| | Age | −1.38 | (−1.65 to −1.11) | −0.557 | <0.001 | |

SUPPLEMENTARY TABLE 1
Association between the presence of the immuno-reactive nephrin
fragments in urine samples of type 2 diabetic patients (all P < 0.0001)

| | | |
|---|---|---|
| N | Y | Total |

25 kDa fragment

| | | | | |
|---|---|---|---|---|
| 50 kDa fragment | N | 240 | 11 | 251 |
| | Y | 53 | 77 | 130 |
| | Total | 293 | 88 | 381 |
| 60 kDa fragment | N | 220 | 6 | 226 |
| | Y | 73 | 82 | 155 |
| | Total | 293 | 88 | 381 |
| 75 kDa fragment | N | 285 | 37 | 322 |
| | Y | 8 | 51 | 59 |
| | Total | 293 | 88 | 381 |

50 kDa fragment

| | | | | |
|---|---|---|---|---|
| 60 kDa fragment | N | 207 | 19 | 226 |
| | Y | 44 | 111 | 155 |
| | Total | 251 | 130 | 381 |
| 75 kDa fragment | N | 250 | 72 | 322 |
| | Y | 1 | 58 | 59 |
| | Total | 251 | 130 | 381 |

60 kDa fragment

| | | | | |
|---|---|---|---|---|
| 75 kDa fragment | N | 226 | 96 | 322 |
| | Y | 0 | 59 | 59 |
| | Total | 226 | 155 | 381 |

SUPPLEMENTARY TABLE 2
Characteristics of Chinese Type 2 diabetic patients according to the
urinary presence of the 25 kDa immuno-reactive nephrin fragment

| | Total | | Yes | | No | | |
|---|---|---|---|---|---|---|---|
| | (n = 381) | | (n = 88) | | (n = 293) | | p-value |
| Gender, n (%) | | | | | | | |
| Male | 198 | (52.0) | 52 | (59.1) | 146 | (49.8) | 0.127 |
| Female | 183 | (48.0) | 36 | (40.9) | 147 | (50.2) | |
| Age, year | 65.7 | (9.3) | 67.0 | (10.3) | 65.3 | (8.99) | 0.122 |
| Diabetes duration, year | 6 | (2-15) | 9 | (2.5-18) | 6 | (2-13) | 0.048 |
| BMI, kg/m² | 25.64 | (4.36) | 25.45 | (4.60) | 25.70 | (4.30) | 0.640 |
| WHR | 0.91 | (0.07) | 0.93 | (0.07) | 0.91 | (0.07) | 0.066 |
| HbA1c, % | 7.19 | (0.99) | 7.32 | (1.15) | 7.16 | (0.94) | 0.181 |
| Systolic BP, mmHg | 132.6 | (12.15) | 133.0 | (13.24) | 132.4 | (11.83) | 0.723 |
| Diastolic BP, mmHg | 78.4 | (6.70) | 78.1 | (7.13) | 78.5 | (6.58) | 0.556 |
| Triglycerides, mmol/L | 1.3 | (1.0-1.9) | 1.4 | (1.0-2.0) | 1.3 | (1.0-1.9) | 0.492 |
| Cholesterol, mmol/L | 5.04 | (0.90) | 4.97 | (0.90) | 5.06 | (0.90) | 0.468 |
| LDL cholesterol, mmol/L | 3.05 | (0.88) | 2.92 | (0.94) | 3.09 | (0.87) | 0.129 |
| HDL cholesterol, mmol/L | 1.23 | (0.35) | 1.20 | (0.34) | 1.24 | (0.35) | 0.391 |
| ACR, mg/g | 22 | (8.9-61.2) | 179.9 | (47.1-763.9) | 14.5 | (7.2-31.5) | 0.249 |
| eGFR, ml/min/1.73 m² | 81.9 | (25.2) | 71.4 | (27.8) | 80.0 | (23.51) | <0.001 |

Data are presented as mean (SD) or median (interquartile range).

SUPPLEMENTARY TABLE 3
Characteristics of normoalbuminuric Chinese Type 2 diabetic patients
according to the urinary presence of the 25 kDa immuno-reactive nephrin fragment

| | Total | | Yes | | No | | |
|---|---|---|---|---|---|---|---|
| | (n = 228) | | (n = 12) | | (n = 216) | | p-value |
| Gender, n (%) | | | | | | | |
| Male | 114 | (50.0) | 7 | (58.3) | 107 | (49.5) | 0.553 |
| Female | 114 | (40.0) | 5 | (41.7) | 109 | (50.5) | |
| Age, year | 64.9 | (9.2) | 67.1 | (10.5) | 64.8 | (9.2) | 0.403 |
| Diabetes duration, yr | 5 | (2-12) | 3.5 | (2-10) | 5.5 | (3-13) | 0.432 |
| BMI, kg/m² | 25.26 | (3.97) | 24.29 | (4.75) | 25.32 | (3.92) | 0.385 |
| WHR | 0.91 | (0.07) | 0.91 | (0.06) | 0.91 | (0.07) | 0.827 |
| HbA1c, % | 7.14 | (0.94) | 7.07 | (1.35) | 7.14 | (0.91) | 0.792 |
| Systolic BP, mmHg | 131.7 | (11.07) | 131.7 | (12.29) | 131.7 | (11.0) | 0.984 |
| Diastolic BP, mmHg | 78.4 | (6.55) | 76.83 | (7.92) | 78.47 | (6.48) | 0.403 |
| Triglycerides, mmol/L | 1.2 | (0.9-1.7) | 1.0 | (0.6-1.5) | 1.2 | (0.9-1.7) | 0.146 |
| Cholesterol, mmol/L | 5.02 | (0.86) | 4.62 | (0.61) | 5.05 | (0.87) | 0.108 |
| LDL cholesterol, mmol/L | 3.09 | (0.86) | 2.84 | (0.55) | 3.10 | (0.87) | 0.312 |
| HDL cholesterol, mmol/L | 1.25 | (0.35) | 1.27 | (0.22) | 1.25 | (0.36) | 0.839 |
| ACR, mg/g | 10.5 | (6.0-16.9) | 15.8 | (11.2-19.5) | 10.3 | (5.9-16.9) | 0.077 |
| eGFR, ml/min/1.73 m² | 85.5 | (22.5) | 65.6 | (25.9) | 86.6 | (21.9) | 0.003 |

Data are presented as mean (SD) or median (interquartile range).

1. A method of detecting type 2 diabetes-related reduced renal function in a mammalian subject comprising detecting one or more nephrin degradation products in a urine sample from the subject, wherein the nephrin degradation product has an apparent molecular weight of about 25 kDa, 50 kDa, 60 kDa and/or 75 kDa, and detection of the nephrin degradation product(s) indicates the presence of type 2 diabetes-related reduced renal function in the subject.

2. The method of claim 1, wherein the subject is a human.

3. The method of claim 1, wherein the subject is normoalbuminuric.

4. (canceled)

5. The method of claim 1, wherein the nephrin degradation products has an apparent molecular weight of about 25 kDa.

6. The method of claim 1, wherein the nephrin degradation products is detected using an antibody.

7. The method of claim 6, wherein the antibody is a monoclonal antibody.

8. The method of claim 1, wherein the mammal exhibits a decline in eGFR of at least 1.0 ml/min/1.73 m².

9. The method of claim 1, wherein the subject with type 2 diabetes has been diagnosed as diabetic for a year or less.

10. The method of claim 1, wherein the nephrin degradation products is detected by ELISA, Western Blotting, RIA, HPLC, SPR, nucleic acid or protein aptamers, SAT, peptide sequencing, and/or MS/MS.

11. The method of claim 1, wherein the nephrin degradation products is detected in a cell free fraction of the urine sample.

12. The method of claim 1, further comprising administering a suitable treatment for reduced renal function, wherein the treatment is one or more antagonists of the renin-angiotensin system (RAS).

13. A method of detecting type 2 diabetes-related reduced renal function in a subject, the method comprising detecting at least one soluble nephrin degradation product with an apparent molecular weight of about 25 kDa, 50 kDa, 60 kDa and/or 75 kDa in a urine sample from the subject using an antibody specific for nephrin, wherein detection of the nephrin degradation products in the urine sample indicates reduced renal function in the subject.

14. The method of claim 13, wherein the subject is human.

15. A kit for detecting type 2 diabetes-related reduced renal function in a mammal comprising an antibody that binds one or more nephrin degradation products with an apparent molecular weight of about 25 kDa, 50 kDa, 60 kDa, and/or 75 kDa in a urine sample and instructions for use.

16. The kit of claim 15, wherein the kit further comprises one or more nephrin degradation products suitable for use as a positive control.

17. The kit of claim 15, wherein the antibody that binds one or more nephrin degradation products is associated with an immunochromatographic device.

18. (canceled)

19. (canceled)

20. (canceled)

21. The method of claim 1, wherein the nephrin degradation product has an apparent molecular weight of about 25 kDa and/or 50 kDa.

22. The method of claim 14, wherein the human is normoalbuminuric.

23. The method of claim 13, wherein the nephrin degradation product has an apparent molecular weight of about 25 KDa and/or 50 kDa.

24. The kit of claim 15, wherein the nephrin degradation product has an apparent molecular weight of about 25 kDa and/or 50 kDa.
